(19) United States
(12) Patent Application Publication
(10) Pub. No.: US 2013/0209358 A1
(43) Pub. Date: Aug. 15, 2013

(54) RADIOTRACER COMPOSITIONS

(71) Applicant: GE HEALTHCARE LIMITED, (US)

(72) Inventors: DAVID JONATHAN BARNETT, STEVENAGE (GB); INGRID HENRIKSEN, OSLO (NO); MARIA CONSTANTINOU, AMERSHAM (GB); ROGER PAUL PETTITT, GREAT MISSENDEN (GB)

(73) Assignee: GE HEALTHCARE LIMITED, LITTLE CHALFONT (GB)

(21) Appl. No.: 13/763,878

(22) Filed: Feb. 11, 2013

Related U.S. Application Data
(60) Provisional application No. 61/597,862, filed on Feb. 13, 2012.

(30) Foreign Application Priority Data
Feb. 13, 2012 (GB) 1202420.4

(51) Int. Cl. A61K 49/00

(52) U.S. Cl. CPC A61K 49/00
USPC 424/1.69

(57) ABSTRACT

The present invention relates to [¹⁸F]-fluciclatide radiopharmaceutical compositions, which are stabilised with a radioprotectant. Also described are methods for the preparation of the radiopharmaceutical compositions, including automated synthesizer methods and cassettes for use in such methods. The invention also includes methods of imaging the mammalian body using the radiopharmaceutical compositions.

### FIELD OF THE INVENTION

The present invention relates to [¹⁸F]-fluciclatide radiopharmaceutical compositions, which are stabilised with a radioprotectant. Also described are methods for the preparation of the radiopharmaceutical compositions, including automated synthesizer methods and cassettes for use in such methods. The invention also includes methods of imaging the mammalian body using the radiopharmaceutical compositions.

### BACKGROUND TO THE INVENTION

Fluciclatide (¹⁸F) is the recommended INN (US Approved Name) for [¹⁸F]-AH111585. [¹⁸F]-AH111585 has been described in both patents and publications, as a PET imaging radiotracer which targets integrin receptors in vivo.

WO 03/006491 discloses compounds of Formula (I):

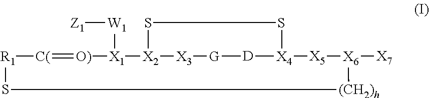

or pharmaceutically acceptable salt thereof
wherein:
G represents glycine
D represents aspartic acid
R₁ represents —(CH₂)ₙ— or —(CH₂)ₙ—C₆H₄— wherein
n represents a positive integer 1 to 10,
h represents a positive integer 1 or 2,
X₁ represents an amino acid residue wherein said amino acid possesses a functional side-chain such as an acid or amine,
X₂ and X₄ represent independently an amino acid residue capable of forming a disulfide bond,
X₃ represents arginine, N-methylarginine or an arginine mimetic,
X₅ represents a hydrophobic amino acid or derivatives thereof,
X₆ represents a thiol-containing amino acid residue,
X₇ is absent or represents a biomodifier moiety,
Z₁ represents an anti-neoplastic agent, a chelating agent or a reporter moiety and
W₁ is absent or represents a spacer moiety.

WO 2006/030291 discloses the synthesis of [¹⁸F]-fluciclatide and radiopharmaceutical compositions containing the same. WO 2006/030291 states that the radiofluorinated peptides of the invention can be prepared rapidly and efficiently, and still have the desired biological activity—of targeting integrin receptors in vivo.

WO 2010/142754 discloses methods of PET imaging of fibrogenesis in the liver in vivo using PET tracers of Formula I:

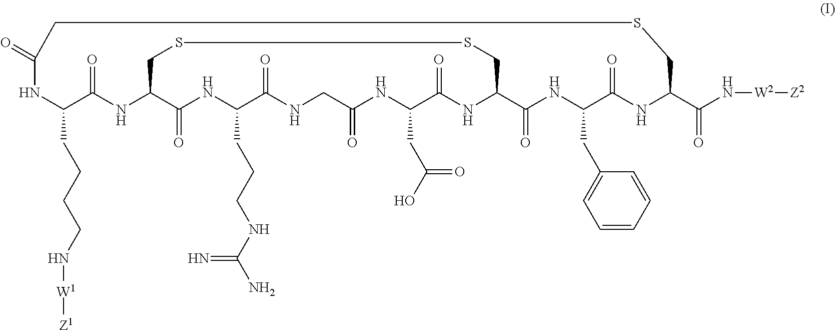

wherein:
one of Z¹ and Z² is a group comprising ¹⁸F, and the other of Z¹ and Z² is H; and,
each of W′ and W² is independently a bivalent linker moiety of Formula Ia:

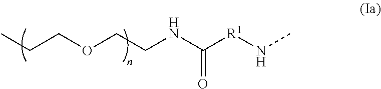

wherein
n is an integer from 1 to 10;
R¹ is C₁₋₅ alkylene, C₂₋₅ oxoalkylene, C₁₋₅ oxaalkylene, or is a C₂₋₅ carbonyl-substituted oxaalkylene; and,
the dotted line represents the point of attachment to either Z¹ or Z².

[¹⁸F]-fluciclatide is a preferred imaging agent described therein. WO 2010/142754 states that the radiopharmaceutical compositions may optionally contain further ingredients such as buffers, pharmaceutically acceptable solubilisers (for example cyclodextrins or surfactants such as Pluronic, Tween, or phospholipids), pharmaceutically acceptable stabilisers or antioxidants (such as ascorbic acid, gentisic acid or para-aminobenzoic acid) or bulking agents for lyophilisation (such as sodium chloride or mannitol).

Glaser et al [Bioconj. Chem., 19(4), 951-957 (2008)], disclose the synthesis and radiolabelling of [¹⁸F]-fluciclatide. Glaser et al state that the radiochemical purity was 96%, and that radio-HPLC analysis of the reaction mixture after 10 minutes incubation indicated almost quantitative coupling efficiency, with only a trace of [¹⁸F]-fluorobenzaldehyde remaining.

[¹⁸F]-fluciclatide has been reported to be useful for imaging breast cancer in human patients [Kenny et al, J. Nucl. Med., 49(6), 879-886 (2008)], as well as for determining changes in tumour vascularity after anti-cancer therapy [Morrison et al, J. Nucl. Med., 50(1), 116-122 (2009)].

Fawdry [Appl. Radiat. Isotop., 65, 1193-1201 (2007)], studied the radiolysis of [¹⁸F]-FDG (fluorodeoxyglucose), and concluded that it can be stabilised with reductant stabilisers chosen from ascorbic acid, sodium thiosulfate, sodium nitrite and iodide. Ascorbic acid, sodium thiosulfate and sodium nitrite were stated to be preferred since they are commercially available to pharmaceutical grade as sterile, pyrogen-free injectable solutions.

Scott et al [Appl. Radiat. Isotop., 67, 88-94 (2009)], reviewed the stability of ¹⁸F radiopharmaceuticals based on N-methyl- or N-dimethyl-substituted aryl amines, and concluded that ethanol, ascorbic acid or nitrones are effective stabilisers.

### The Present Invention.

The present inventors have found that [¹⁸F]-fluciclatide, when prepared as described in the literature suffers from previously unrecognised problems:
(i) radioactive instability at higher radioactive concentration (RAC)—meaning that the number of patient doses available from a given radioactive synthesis is limited. This means that the radioactive synthesis must be carried out repeatedly when multiple doses are required;
(ii) insufficient radiochemical purity (RCP) at longer times after synthesis—thus limiting the usable clinical imaging shelf-life post synthesis.

There is therefore a need for [¹⁸F]-fluciclatide compositions which exhibit higher stability and RCP.

The present invention provides improved [¹⁸F]-fluciclatide radiopharmaceutical compositions which exhibit more reproducible initial radiochemical purity (RCP) and improved stability post-synthesis, so that an RCP of >95% is maintained at 8 hours post-synthesis. The problem of unsatisfactory RCP for [¹⁸F]-fluciclatide preparations under certain conditions of radioactivity levels, radioactive concentrations or reconstitution volumes was not recognised in the prior art.

The positron-emitting radioisotope ¹⁸F has a half-life of 110 minutes. In order to have a minimum of either 9 patient doses at 2-hours after EOS (End of Synthesis), or 2 such doses 6-hours after EOS, the initial RAC must be up to 500 MBq/mL. The present invention provides such a formulation for [¹⁸F]-fluciclatide, by using the radioprotectant para-aminobenzoic acid (i.e. 4-aminobenzoic acid; pABA) or biocompatible salts thereof. In fact, the present invention permits [¹⁸F]-fluciclatide preparations having an RAC of up to 860 MBq/mL at EOS.

The present inventors have also established that the well-known radioprotectant ascorbic acid/ascorbate is not ideal for [¹⁸F]-fluciclatide preparations. That is because, for ascorbic acid the pH of the preparation is outside physiological pH, as described by Scott et al (above). Scott et al concluded that sodium ascorbate is an acceptable alternative. The present inventors have, however, established that sodium ascorbate is not ideal for automated synthesizer preparations, since it undergoes rapid oxidation in aqueous solution—so may need to be used in lyophilized form or with a further stabiliser or be freshly dissolved prior to use. That presents shelf-life issues for commercial radiotracer synthesis.

The present inventors have also established that the prior art radioprotectant ethanol is ineffective as stabilising [¹⁸F]-fluciclatide preparations. Thus, [¹⁸F]-fluciclatide is prepared in 7% aqueous ethanol, but such preparations still exhibit radiolysis.

The radioprotectants of the present invention also have the advantage that they have radiostabilising properties at acidic pH, and can thus be incorporated as in-process stabilisers for the aminooxy-peptide ¹⁸F-fluorobenzaldehyde conjugation reaction (which is carried out at pH 2.6 to 3.0).

### DESCRIPTION OF THE FIGURES

FIG. 1 shows the decrease in Radiochemical Purity (RCP) of [¹⁸F]-Fluciclatide with and without Na-pABA as a function of the radioactive concentration at end of synthesis (EOS).

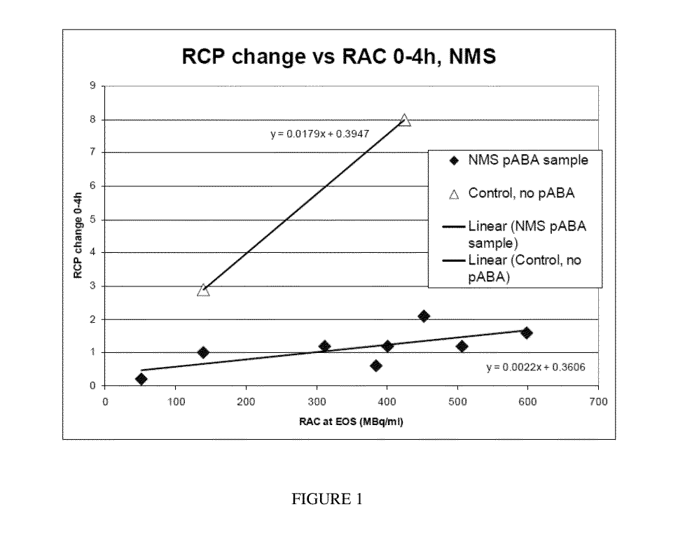

### DETAILED DESCRIPTION OF THE INVENTION

In a first aspect, the present invention provides a radiopharmaceutical composition which comprises:
(i) fluciclatide;
(ii) a radioprotectant chosen from para-aminobenzoic acid or a salt thereof with a biocompatible cation;
in a biocompatible carrier in a form suitable for mammalian administration;
where [¹⁸F]-fluciclatide is the compound of Formula I:

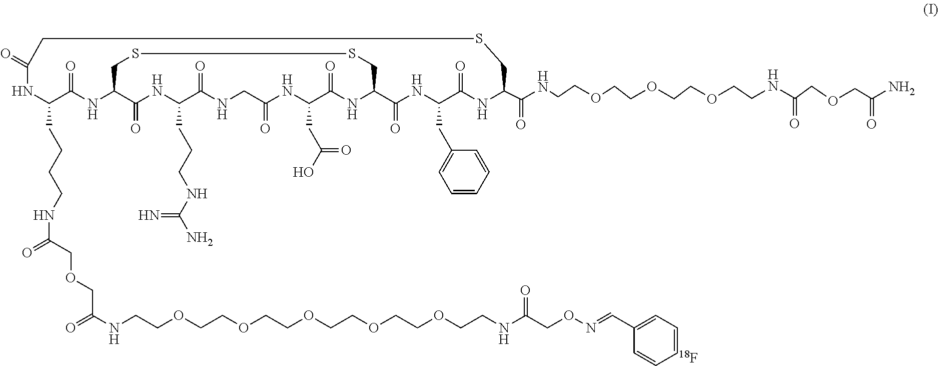

The term “radiopharmaceutical” has its conventional meaning, and refers to a radioactive compound suitable for in vivo mammalian administration for use in diagnosis or therapy.

By the term “radioprotectant” is meant a compound which inhibits degradation reactions, such as redox processes, by trapping highly-reactive free radicals, such as oxygen-containing free radicals arising from the radiolysis of water. The radioprotectant of the present invention is suitably chosen from para-aminobenzoic acid (i.e. 4-aminobenzoic acid) and salts thereof with a biocompatible cation. These radioprotectants are commercially available, including in pharmaceutical grade purity. For para-aminobenzoic acid and sodium para-aminobenzoate, a suitable concentration range is 0.5 to 4.0, preferably 1.0 to 3.0, more preferably 1.5 to 2.5, most preferably 1.8 to 2.2 mg/mL. 2.0 mg/mL is especially preferred.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The “biocompatible carrier” is a fluid, especially a liquid, in which the radiopharmaceutical can be suspended or preferably dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous buffer solution comprising a biocompatible buffering agent (e.g. phosphate buffer); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). Preferably the biocompatible carrier is pyrogen-free water for injection, isotonic saline or phosphate buffer.

By the phrase “in a form suitable for mammalian administration” is meant a composition which is sterile, pyrogen-free, lacks compounds which produce toxic or adverse effects, and is formulated at a biocompatible pH (approximately pH 4.0 to 10.5, preferably 6.5 to 9.5 for the agents of the present invention) and physiologically compatible osmolality. Such compositions lack particulates which could risk causing emboli in vivo, and are formulated so that precipitation does not occur on contact with biological fluids (e.g. blood). Such compositions also contain only biologically compatible excipients, and are preferably isotonic.

Preferably, the mammal is an intact mammalian body in vivo, and is more preferably a human subject. Preferably, the radiopharmaceutical can be administered to the mammalian body in a minimally invasive manner, i.e. without a substantial health risk to the mammalian subject even when carried out under professional medical expertise. Such minimally invasive administration is preferably intravenous administration into a peripheral vein of said subject, without the need for local or general anaesthetic.

The term “comprising” has its conventional meaning throughout this application and implies that the composition must have the components listed, but that other, unspecified compounds or species may be present in addition. The term ‘comprising’ includes as a preferred subset “consisting essentially of” which means that the composition has the components listed without other compounds or species being present.

The radiopharmaceutical composition of the present invention is suitably provided in a container wherein the headspace gas contains 5 to 30%, preferably 10-25%, most preferably 18-22% oxygen. Ideally, the headspace gas is air. Thus, the present inventors have found that when pure nitrogen is used as the headspace gas, there is more variation in RCP.

The radiopharmaceutical composition may contain additional optional excipients such as: an antimicrobial preservative, pH-adjusting agent, filler, solubiliser or osmolality adjusting agent.

By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, i.e. methyl, ethyl, propyl or butyl paraben or mixtures thereof; benzyl alcohol; ethanol, phenol; cresol; cetrimide and thiomersal. Preferred antimicrobial preservative(s) are the parabens or ethanol.

The term “pH-adjusting agent” means a compound or mixture of compounds useful to ensure that the pH of the composition is within acceptable limits (approximately pH 4.0 to 10.5, preferably 6.5 to 9.5 for the agents of the present invention) for human or mammalian administration. Suitable such pH-adjusting agents include pharmaceutically acceptable buffers, such as tricine, phosphate, acetate or TRIS [i.e. tris(hydroxymethyl)aminomethane], and pharmaceutically acceptable bases such as sodium carbonate, sodium bicarbonate or mixtures thereof.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

By the term “solubiliser” is meant an additive present in the composition which increases the solubility of the radiopharmaceutical in the solvent. A preferred such solvent is aqueous media, and hence the solubiliser preferably improves solubility in water. Suitable such solubilisers include: C₁₋₄ alcohols; glycerine; polyethylene glycol (PEG); propylene glycol; polyoxyethylene sorbitan monooleate; sorbitan monooloeate; polysorbates (e.g. Tween™); poly(oxyethylene)poly(oxypropylene)poly(oxyethylene) block copolymers (Pluronics™); cyclodextrins (e.g. alpha, beta or gamma cyclodextrin, hydroxypropyl-β-cyclodextrin or hydroxypropyl-γ-cyclodextrin) and lecithin.

Preferred solubilisers are cyclodextrins, C₁₋₄ alcohols, polysorbates and Pluronics™, more preferably cyclodextrins and C₂₋₄ alcohols. When the solubiliser is an alcohol, it is preferably ethanol or propanol, more preferably ethanol. Ethanol has potentially a dual role, since it can also function as a biocompatible carrier and as an antimicrobial preservative. When the solubiliser is a cyclodextrin, it is preferably a gamma cyclodextrin, more preferably hydroxypropyl-β-cyclodextrin (HPCD). The concentration of cyclodextrin can be from about 0.1 to about 40 mg/ml, preferably between about 5 and about 35 mg/ml, more preferably 20 to 30 mg/ml, most preferably around 25 mg/ml.

### Preferred Features.

The RAC of the radiopharmaceutical composition of the first aspect at EOS is preferably in the range 100-860, more preferably 200-700, most preferably 250-600 MBq/mL.

The radioprotectant of the present invention preferably comprises sodium para-aminobenzoate. An additional radioprotectant may also optionally be present. More preferably, the radioprotectant of the present invention consists essentially of para-aminobenzoic acid or a salt thereof with a biocompatible cation. Most preferably, the radioprotectant of the present invention consists essentially of sodium para-aminobenzoate.

Preferably, the grade of radioprotectant used is pharmaceutical grade. Thus, technical grade material has been shown to give rise to additional chemical impurities in the radiopharmaceutical composition.

The radiopharmaceutical composition of the first aspect is suitably provided in a pharmaceutical grade container. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity.

The radiopharmaceutical composition of the first aspect may also be provided in a syringe. Pre-filled syringes are designed to contain a single human dose, or “unit dose” and are therefore preferably a single-use or other syringe suitable for clinical use. The radiopharmaceutical syringe is preferably provided with a syringe shield to minimise radiation dose to the operator.

Pharmaceutical grade pABA and sodium para-aminobenzoate are commercially available, and can be obtained from e.g. Sigma or Merck. [¹⁸]-fluciclatide and [¹⁸F]-fluciclatide compositions can be obtained as described in the second aspect (below).

In a second aspect, the present invention provides a method of preparation of the radiopharmaceutical composition of the first aspect, which comprises:
(i) reaction of a precursor with a supply of [¹⁸F]fluoride in the presence of a radioprotectant; or
(ii) reaction of a precursor with a supply of [¹⁸F]fluoride to give [¹⁸F]-fluciclatide, followed by the addition of a radioprotectant to said [¹⁸F]-fluciclatide; or
(iii) addition of a radioprotectant to [¹⁸F]-fluciclatide; or
(iv) combinations of (i), (ii) and (iii),
   wherein said radioprotectant is chosen from para-aminobenzoic acid or a salt thereof with a biocompatible cation, [¹⁸F]-fluciclatide is as defined in the first aspect; and wherein said precursor is of Formula II:

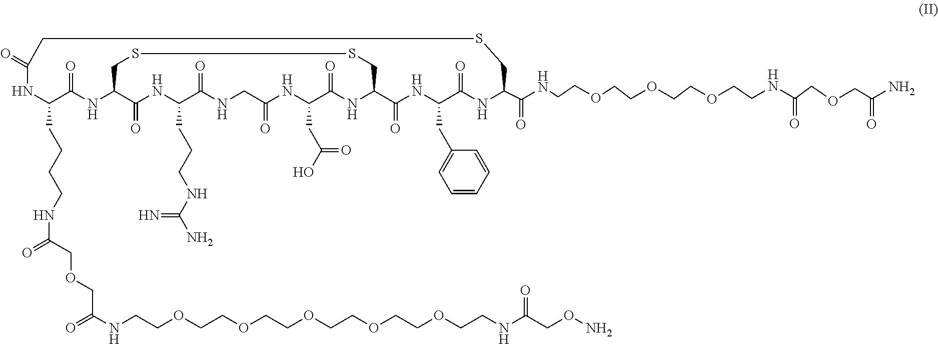 [SMILES: N=C(N)NCCC[C@@H]1NC(=O)[C@@H]2CSSC[C@H](NC(=O)[C@H](CC(=O)O)NC(=O)CNC1=O)C(=O)N[C@@H](Cc1ccccc1)C(=O)N[C@H](C(=O)NCCOCCOCCOCCNC(=O)COCC(N)=O)CSCC(=O)N[C@@H](CCCCNC(=O)COCC(=O)NCCOCCOCCOCCOCCOCCNC(=O)CON)C(=O)N2]

Preferred aspects of the radioprotectant in the second aspect are as described in the first aspect (above).

The term “combinations of (i), (ii) and (iii)” in option (iv), refers to the possibility of adding the radioprotectant in portions at different phases of the preparation of the radiotracer. Option (i) is preferred, since having the radioprotectant present from the outset helps to minimise radiolysis during the synthesis.

The precursor of Formula II is non-radioactive. It can be prepared as described by Indrevoll et al [Bioorg. Med. Chem. Lett., 16, 6190-6193 (2006)] and in the present Examples.

The supply of [¹⁸F]fluoride may either be:
(i) delivered directly from a cyclotron and formulated using an ion exchange cartridge and appropriate eluent or
(ii) in the form of GMP [¹⁸F]NaF produced on an automated platform in a GMP facility.

The production of [¹⁸F]fluoride suitable for radiopharmaceutical applications is well-known in the art, and has been reviewed by Hjelstuen et al [Eur. J. Pharm. Biopharm., 78(3), 307-313 (2011)], and Jacobson et al [Curr. Top. Med. Chem., 10(11), 1048-1059 (2010)]. [¹⁸F]NaF can be produced using an “automated synthesizer” as described below.

The method of the second aspect is preferably carried out using an automated synthesizer apparatus. By the term “automated synthesizer” is meant an automated module based on the principle of unit operations as described by Satyamurthy et al [Clin. Positr. Imag., 2(5), 233-253 (1999)]. The term ‘unit operations’ means that complex processes are reduced to a series of simple operations or reactions, which can be applied to a range of materials. Such automated synthesizers are preferred for the method of the present invention especially when a radiopharmaceutical composition is desired. They are commercially available from a range of suppliers [Satyamurthy et al, above], including: GE Healthcare; CTI Inc; Ion Beam Applications S.A. (Chemin du Cyclotron 3, B-1348 Louvain-La-Neuve, Belgium); Raytest (Germany) and Bioscan (USA).

Commercial automated synthesizers also provide suitable containers for the liquid radioactive waste generated as a result of the radiopharmaceutical preparation. Automated synthesizers are not typically provided with radiation shielding, since they are designed to be employed in a suitably configured radioactive work cell. The radioactive work cell provides suitable radiation shielding to protect the operator from potential radiation dose, as well as ventilation to remove chemical and/or radioactive vapours.

The automated synthesizer preferably comprises a cassette. By the term “cassette” is meant a piece of apparatus designed to fit removably and interchangeably onto an automated synthesizer apparatus (as defined above), in such a way that mechanical movement of moving parts of the synthesizer controls the operation of the cassette from outside the cassette, i.e. externally. Suitable cassettes comprise a linear array of valves, each linked to a port where reagents or vials can be attached, by either needle puncture of an inverted septum-sealed vial, or by gas-tight, marrying joints. Each valve has a male-female joint which interfaces with a corresponding moving arm of the automated synthesizer. External rotation of the arm thus controls the opening or closing of the valve when the cassette is attached to the automated synthesizer. Additional moving parts of the automated synthesizer are designed to clip onto syringe plunger tips, and thus raise or depress syringe barrels.

The cassette is versatile, typically having several positions where reagents can be attached, and several suitable for attachment of syringe vials of reagents or chromatography cartridges (e.g. solid phase extraction or SPE). The cassette always comprises a reaction vessel. Such reaction vessels are preferably 0.5 to 10 mL, more preferably 0.5 to 5 mL and most preferably 0.5 to 4 mL in volume and are configured such that 3 or more ports of the cassette are connected thereto, to permit transfer of reagents or solvents from various ports on the cassette. Preferably the cassette has 15 to 40 valves in a linear array, most preferably 20 to 30, with 25 being especially preferred. The valves of the cassette are preferably each identical, and most preferably are 3-way valves. The cassettes are designed to be suitable for radiopharmaceutical manufacture and are therefore manufactured from materials which are of pharmaceutical grade and ideally also are resistant to radiolysis.

Preferred automated synthesizers of the present invention comprise a disposable or single use cassette which comprises all the reagents, reaction vessels and apparatus necessary to carry out the preparation of a given batch of radiofluorinated radiopharmaceutical. The cassette means that the automated synthesizer has the flexibility to be capable of making a variety of different radiopharmaceuticals with minimal risk of cross-contamination, by simply changing the cassette. The cassette approach also has the advantages of: simplified set-up hence reduced risk of operator error; improved GMP (Good Manufacturing Practice) compliance; multi-tracer capability; rapid change between production runs; pre-run automated diagnostic checking of the cassette and reagents; automated barcode cross-check of chemical reagents vs the synthesis to be carried out; reagent traceability; single-use and hence no risk of cross-contamination, tamper and abuse resistance. A preferred cassette of the invention is described in the third aspect (below).

In a third aspect, the present invention provides a single use, cassette which comprises either:
(i) separate supplies of a precursor and a radioprotectant; or
(ii) a precursor and a radioprotectant, provided together as a composition;
   wherein said radioprotectant is chosen from para-aminobenzoic acid or a salt thereof with a biocompatible cation, and wherein said precursor is of Formula II:

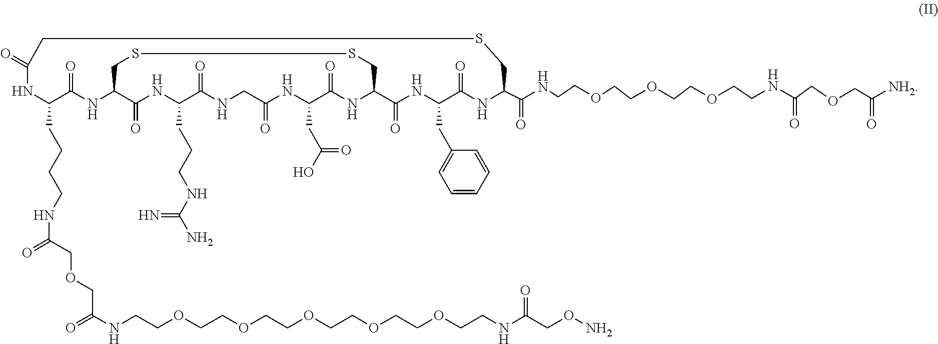 [SMILES: N=C(N)NCCC[C@@H]1NC(=O)[C@@H]2CSSC[C@H](NC(=O)[C@H](CC(=O)O)NC(=O)CNC1=O)C(=O)N[C@@H](Cc1ccccc1)C(=O)N[C@H](C(=O)NCCOCCOCCOCCNC(=O)COCC(N)=O)CSCC(=O)N[C@@H](CCCCNC(=O)COCC(=O)NCCOCCOCCOCCOCCOCCNC(=O)CON)C(=O)N2]

Preferred aspects of the radioprotectant and automated synthesizer in the third aspect are as described in the first and second aspects respectively (above). The radioprotectant is preferably sodium para-aminobenzoate.

The cassette preferably comprises the radioprotectant which is provided as a solution. The solvent for such solutions is preferably a biocompatible carrier as described above. Such solutions are preferably stored in the dark.

In a fourth aspect, the present invention provides a method of stabilising a [¹⁸F]-fluciclatide radiopharmaceutical composition which comprises the use of para-aminobenzoic acid or a salt thereof with a biocompatible cation. In the method of the fourth aspect, the radioprotectant is preferably sodium para-aminobenzoate.

In a fifth aspect, the present invention provides a method of imaging of the mammalian body which comprises imaging a mammal which had previously been administered with the radiopharmaceutical composition of the first aspect. Preferred aspects of the radiopharmaceutical composition in the seventh aspect are as described in the first aspect (above). Preferably, the mammal is an intact mammalian body in vivo, and is more preferably a human subject.

The imaging of the fifth aspect is preferably to image a mammalian subject suffering from a disease in which in which integrins are abnormally expressed, such as angiogenesis, fibrosis or inflammation. The method of imaging of the fifth aspect preferably comprises PET (Positron Emission Tomography).

In a sixth aspect, the present invention provides a method of diagnosis of the mammalian body which comprises the method of imaging of the fifth aspect. Preferably, the mammal is an intact mammalian body in vivo, and is more preferably a human subject. Preferred aspects of the method of imaging in the sixth aspect are as described in the fifth aspect (above). Preferred aspects of the radiopharmaceutical composition in the sixth aspect are as described in the first aspect (above).

The invention is illustrated by the non-limiting Examples detailed below. Example 1 provides the synthesis of Precursor 1 of the invention. Example 2 provides the synthesis of [¹⁸F]-FBA, and Example 3 the purification of [¹⁸F]-FBA to obtain compositions of the invention. Example 4 provides the synthesis of Compound 1 of the invention. Example 5 demonstrates the effectiveness of the radioprotectant formulations of the invention on the RCP of Compound 1, compared with prior art (i.e. unstabilised formulations). It can be seen that the prior art formulations have unsatisfactory RCP at 2 to 4 hours post preparation, even in aqueous ethanol. Example 6 describes the stabilising effects at different concentrations of radioprotectant. Example 7 shows that the addition of a radioprotectant is unlikely to effect the clinical imaging efficacy of [¹⁸F]-fluciclatide.

### ABBREVIATIONS

Conventional single letter or 3-letter amino acid abbreviations are used.
Ac: Acetyl.
ACN: Acetonitrile.
Boc: tert-Butyloxycarbonyl.
DIPEA: N,N-diisopropylethylamine.
DMAB: 4-(dimethylamino)benzaldehyde.
DMSO: Dimethylsulfoxide.
EOS: End of synthesis.
FBA: 4-Fluorobenzaldehyde.
Fmoc: 9-Fluorenylmethoxycarbonyl.
HATU: O-(7-Azabenzotriazol-1-yl)-N,N,N′,N′-tetramethyluronium hexafluorophosphate.
HBA: 4-hydroxybenzaldehyde.
HPLC: High performance liquid chromatography.
MCX Mixed mode cation exchange cartridge
Na-pABA: sodium para-aminobenzoate.
NMM: N-methymorpholine.
NMP: 1-Methyl-2-pyrrolidinone.
PBS: Phosphate-buffered saline.
PET: Positron Emission Tomography.
PyBOP: Benzotriazol-1-yl-oxytripyrrolidinophosphonium hexafluorophosphate.
RAC: radioactive concentration.
RCP: Radiochemical purity.
RT: room temperature.
SPE: solid-phase extraction.
tBu: tert-Butyl.
TFA: Trifluoroacetic acid.
TFP: Tetrafluorophenyl.
TMAB: 4-(trimethylammonium)benzaldehyde.
T_{R}: retention time.

TABLE 1
Compounds of the Invention.

| Name | Structure |
|---|---|
| Peptide 1 | 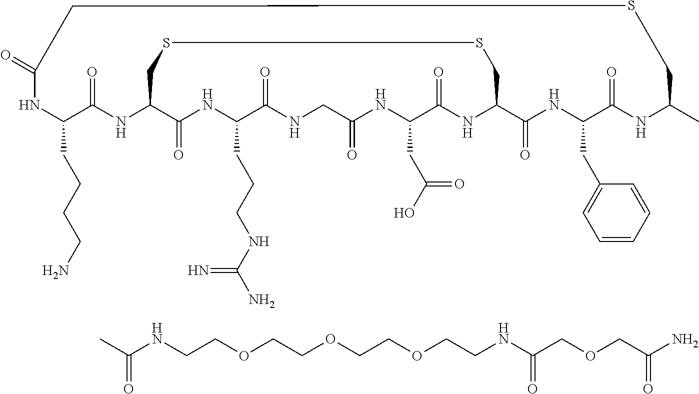 |
| Precursor 1 | 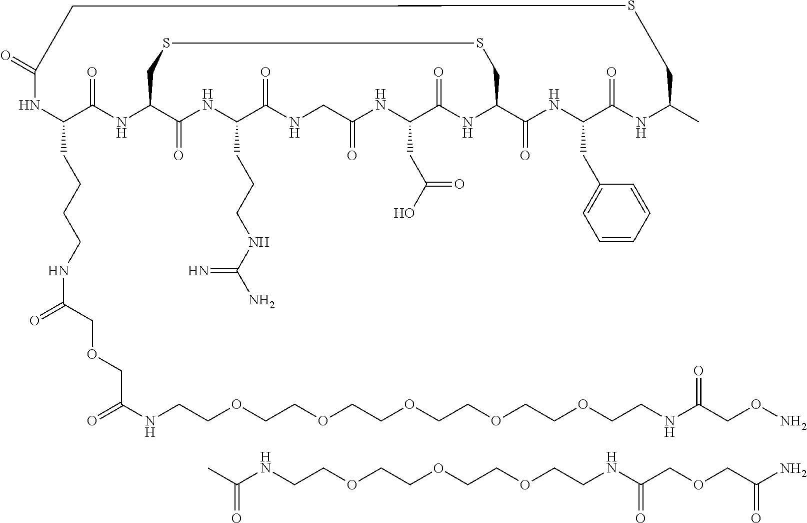 |
| Compound 1 | 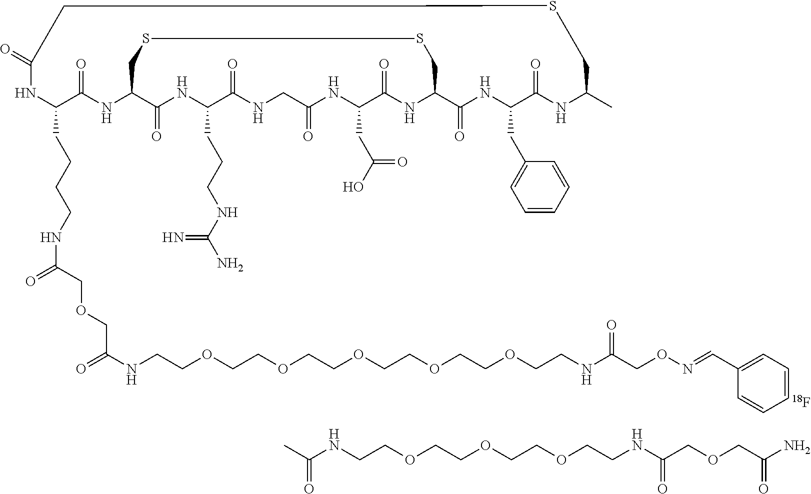 |

### Example 1

### Synthesis of Precursor 1

Peptide 1 was synthesised using standard peptide synthesis, as described by Indrevoll et al [Bioorg. Med. Chem. Lett., 16, 6190-6193 (2006)].

### (a) 1,17-Diazido-3,6,9,12,15-pentaoxaheptadecane

A solution of dry hexaethylene glycol (25 g, 88 mmol) and methanesulfonyl chloride (22.3 g, 195 mmol) in dry THF (125 mL) was kept under argon and cooled to 0° C. in an ice/water bath. A solution of triethylamine (19.7 g, 195 mmol) in dry THF (25 mL) was added dropwise over 45 min. After 1 hr the cooling bath was removed and the reaction was stirred for another for 4 hrs. Water (55 mL) was then added to the mixture, followed by sodium hydrogencarbonate (5.3 g, to pH 8) and sodium azide (12.7 g, 195 mmol). THF was removed by distillation and the aqueous solution was refluxed for 24 h (two layers were formed). The mixture was cooled, ether (100 mL) was added and the aqueous phase was saturated with sodium chloride. The phases were separated and the aqueous phase was extracted with ether (4×50 mL). The combined organic phases were washed with brine (2×50 mL) and dried (MgSO₄). Filtration and evaporation of the solvent gave a yellow oil 26 g (89%). The product was used in the next step without further purification.

### (b) 17-Azido-3,6,9,12,15-pentaoxaheptadecanamine

To a vigorously stirred suspension of 1,17-diazido-3,6,9,12,15-pentaoxaheptadecane (25 g, 75 mmol) in 5% HCl (200 mL) was added a solution of triphenylphosphine (19.2 g, 73 mmol) in ether (150 mL) over 3 hrs at room temperature. The reaction mixture was stirred for additional 24 hrs. The phases were separated and the aqueous phase was extracted with dichloromethane (3×40 mL). The aqueous phase was cooled in an ice/water bath and the pH was adjusted to 12 by addition of solid potassium hydroxide. The aqueous phase was concentrated and the product was taken up in dichloromethane (150 mL). The organic phase was dried (Na₂SO₄) and concentrated giving a yellow oil 22 g (95%). The product was identified by electrospray mass spectrometry (ESI-MS) (MH+ calculated: 307.19. found 307.4). The crude oil was used in the next step without further purification.

### (c) 23-Azido-5-oxo-6-aza-3,9,12,15,18,21-hexaoxatricosanoic acid

To a solution of 17-azido-3,6,9,12,15-pentaoxaheptadecanamine (15 g, 50 mmol) in dichloromethane (100 mL) was added diglycolic anhydride (Acros, 6.4 g, 55 mmol). The reaction mixture was stirred overnight. The reaction was monitored by ESI-MS analysis, and more reagents were added to drive the reaction to completion. The solution was concentrated to give a yellow residue which was dissolved in water (250 mL). The product was isolated from the aqueous phase by continuous extraction with dichloromethane overnight. Drying and evaporation of the solvent gave a yield of 18 g (85%). The product was characterized by ESI-MS analysis (MH+ calculated: 423.20. found 423.4). The product was used in the next step without further purification.

### (d) 23-Amino-5-oxo-6-aza-3,9,12,15,18,21-hexaoxatricosanoic acid

23-Azido-5-oxo-6-aza-3,9,12,15,18,21-hexaoxatricosanoic acid (9.0 g, 21 mmol) was dissolved in water (50 mL) and reduced using H₂(g)-Pd/C (10%). The reaction was run until ESI-MS analysis showed complete conversion to the desired product (MH+ calculated: 397.2. found 397.6). The crude product was used in the next step without further purification.

### (e) (Boc-aminooxy)acetyl-PEG(6)-diglycolic acid

A solution of dicyclohexycarbodiimide (515 mg, 2.50 mmol) in dioxan (2.5 mL) was added dropwise to a solution of (Boc-aminooxy)acetic acid (477 mg, 2.50 mmol) and N-hydroxysuccinimide (287 mg, 2.50 mmol) in dioxan (2.5 mL). The reaction was stirred at RT for 1 h and filtered. The filtrate was transferred to a reaction vessel containing a solution of 23-amino-5-oxo-6-aza-3,9,12,15,18,21-hexaoxatricosanoic acid (1.0 g, 2.5 mmol) and NMM (278 μl, 2.50 mmol) in water (5 mL). The mixture was stirred at RT for 30 min. ESI-MS analysis showed complete conversion to the desired product (MH+ calculated: 570.28. found 570.6). The crude product was purified by preparative HPLC (column: Phenomenex Luna 5μ C18 (2) 250×21.20 mm, detection: 214 nm, gradient: 0-50% B over 60 min where A=H₂O/0.1% TFA and B=acetonitrile/0.1% TFA, flow rate: 10 mL/min) affording 500 mg (38%) of pure product. The product was analyzed by HPLC (column: Phenomenex Luna 3μ C18 (2), 50×2.00 mm, detection: 214 nm, gradient: 0-50% B over 10 min where A=H₂O/0.1% TFA and B=acetonitrile/0.1% TFA, flow rate: 0.75 mL/min, Rt=5.52 min). Further confirmation was carried out by NMR analysis.

### (f) Conjugation of (Boc-aminooxy)acetyl-PEG(6)-diglycolic acid to Peptide 1

(Boc-aminooxy)acetyl-PEG(6)-diglycolic acid (0.15 mmol, 85 mg) and PyAOP (0.13 mmol, 68 mg) were dissolved in DMF (2 mL). NMM (0.20 mmol, 20 μL) was added and the mixture was stirred for 10 min. A solution of Peptide 1 (0.100 mmol, 126 mg) and NMM (0.20 mmol, 20 μL) in DMF (4 mL) was added and the reaction mixture was stirred for 25 min. Additional NMM (0.20 mmol, 20 μL) was added and the mixture was stirred for another 15 min. DMF was evaporated in vacuo and the product was taken up in 10% acetonitrile-water and purified by preparative HPLC (column: Phenomenex Luna 5μ C18 (2) 250×21.20 mm, detection: UV 214 nm, gradient: 5-50 B over 40 min where A=H₂O/0.1% TFA and B=acetonitrile/0.1% TFA, flow rate: 10 mL/min) affording 100 mg semi-pure product. A second purification step where TFA was replaced by HCOOH (gradient: 0-30% B, otherwise same conditions as above) afforded 89 mg (50%). The product was analysed by HPLC (column: Phenomenex Luna 3μ C18 (2) 50×2 mm, detection: UV 214 nm, gradient: 0-30% B over 10 min where A=H₂O/0.1% HCOOH and B=acetonitrile/0.1% HCOOH, flow rate: 0.3 mL/min, Rt: 10.21 min). Further product characterisation was carried out using ESI-MS (MH22+ calculated: 905.4. found: 906.0).

### (g) Deprotection

Deprotection was carried out by addition of TFA containing 5% water to 10 mg of peptide.

### Example 2

### Radiosynthesis of [¹⁸F]-Fluorobenzaldehyde (¹⁸F-FBA)

[¹⁸F]-fluoride was produced using a GEMS PETtrace cyclotron with a silver target via the [¹⁸O](p,n) [¹⁸F] nuclear reaction. Total target volumes of 1.5-3.5 mL were used. The radiofluoride was trapped on a Waters QMA cartridge (pre-conditioned with carbonate), and the fluoride is eluted with a solution of Kryptofix_{2.2.2} (4 mg, 10.7 μM) and potassium carbonate (0.56 mg, 4.1 μM) in water (80 μL) and acetonitrile (320 μL). Nitrogen was used to drive the solution off the QMA cartridge to the reaction vessel. The [¹⁸F]-fluoride was dried for 9 minutes at 120° C. under a steady stream of nitrogen and vacuum. Trimethylammonium benzaldehyde triflate, [Haka et al, J. Lab. Comp. Radiopharm., 27, 823-833 (1989)] (3.3 mg, 10.5 μM), in DMSO (1.1 mL) was added to the dried [¹⁸F]-fluoride, and the mixture heated at 105° C. for 7 minutes to produce 4[¹⁸F]-fluorobenzaldehyde.

### Example 3

### Purification of [¹⁸F]-Fluorobenzaldehyde (¹⁸F-FBA)

The crude labelling mixture from Example 2 was diluted with ammonium hydroxide solution and loaded onto an MCX+SPE cartridge (pre-conditioned with water as part of the FASTlab sequence). The cartridge was washed with water, dried with nitrogen gas before elution of 4-[¹⁸F]-fluorobenzaldehyde back to the reaction vessel in ethanol (1.8 mL). A total volume of ethanol of 2.2 mL was used for the elution but the initial portion (0.4 mL) was discarded as this did not contain [¹⁸F]-FBA. 4-7% (decay corrected) of the [¹⁸F] radioactivity remained trapped on the cartridge.

### Example 4

### Preparation of [¹⁸F]-fluciclatide (Compound 1)

The conjugation of [¹⁸F]-FBA with Precursor 1 (5 mg) was performed in a solution of ethanol (1.8 mL) and water (1.8 mL) in the presence of aniline hydrochloride. The reaction mixture was maintained at 60° C. for 5 minutes.

### Example 5

### Effect of Radioprotectant on [¹⁸F]-fluciclatide (Compound 1)

[¹⁸F]-Fluciclatide was prepared with and without sodium para-aminobenzoate radioprotectant, at different RAC values, and the RCP determined (by HPLC) at 0, 2 and 4 hours post preparation. The results are summarised in Table 1:

TABLE 1

| | RAC (at EOS) | RCP (%) | | |
|---|---|---|---|---|
| Agent | MBq/mL | 0 hours | 2 hours | 4 hours |
| Compound 1 | 227 | 98 | 87 | 84 |
| (no radioprotectant) | 373 | 100 | 80 | 78 |
| | 492 | 88 | 77 | 73 |
| Compound 1 | 251 | 100 | 99 | 97 |
| (pABA 0.66 mg/mL) | | | | |
| Compound 1 | 402 | 100 | 97 | 95 |
| (pABA 0.34 mg/mL) | | | | |

### Example 6

### Optimising the Concentration of Radioprotectant

Example 5 was repeated using sodium para-aminobenzoate (Na-pABA). The results are summarised in Table 2:

TABLE 2

| Na—pABA Conc. | RAC (at EOS) | RCP (%) | | | |
|---|---|---|---|---|---|
| (mg/mL) | MBq/mL | 0 h | 2 h | 4 h | 6 h |
| 0.38 | 297 | 99 | 96 | 95 | n.a. |
| 0.50 | 302 | 100 | 98 | 97 | n.a. |
| 0.77 | 345 | 100 | 98 | 97 | n.a. |
| 0.77 | 445 | 98 | 97 | 93 | n.a. |
| 1.34 | 206 | 100 | 100 | 99 | n.a. |
| 1.34 | 224 | 100 | 100 | 100 | n.a. |
| 1.55 | 139 | 98 | 98 | 97 | 97 |
| 1.55 | 545 | 100 | 98 | 96 | n.a. |
| 1.75 | 579 | 100 | 98 | 96 | n.a. |
| 1.90 | 312 | 99 | 98 | 98 | 97 |
| 1.96 | 401 | 99 | 99 | 98 | 99 |
| 2.01 | 598 | 99 | 98 | 97 | 97 |
| 2.26 | 452 | 99 | 97 | 97 | 96 |

n.a. = not available.

### Example 7

### Biodistribution

The biodistribution of the radioprotectant-stabilised formulations of the present invention was compared with that of unstabilised [¹⁸F]-fluciclatide in normal mice, and in the LLC tumour model.

pABA had no effect on the biodistribution of radioactivity following intravenous administration of [¹⁸F] fluciclatide (Compound 1). In addition, the current study demonstrated that addition of pABA to the [¹⁸F] fluciclatide formulation had no effect on the biodistribution of radioactivity to LLC tumours, which are known to be highly angiogenic.

1. A radiopharmaceutical composition which comprises:
(i) [¹⁸F]-fluciclatide;
(ii) a radioprotectant chosen from para-aminobenzoic acid or a salt thereof with a biocompatible cation;
in a biocompatible carrier in a form suitable for mammalian administration;
where [¹⁸F]-fluciclatide is the compound of Formula I:

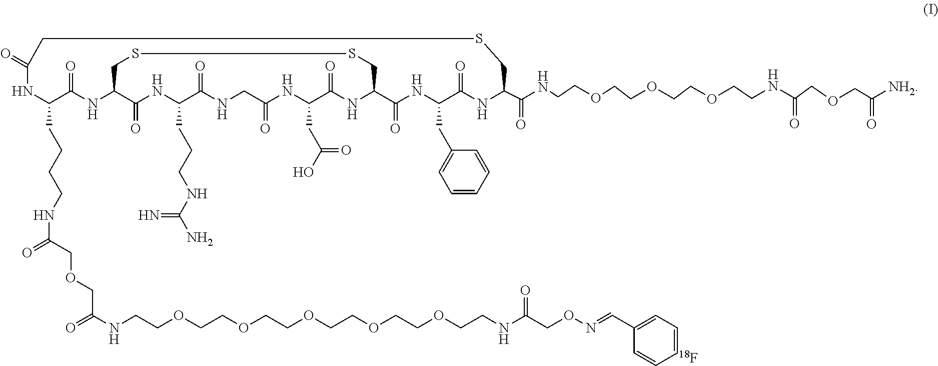

2. The radiopharmaceutical composition of claim 1, where the radioprotectant is sodium para-aminobenzoate.

3. The radiopharmaceutical composition of claim 1, where the radiopharmaceutical is provided in a syringe.

4. The radiopharmaceutical composition of claim 1, where the radiopharmaceutical is provided in a vial fitted with a closure.

5. A method of preparation of the radiopharmaceutical composition of claim 1, which comprises:
(i) reaction of a precursor with a supply of [¹⁸F]fluoride in the presence of a radioprotectant; or
(ii) reaction of a precursor with a supply of [¹⁸F]fluoride to give [¹⁸F]-fluciclatide, followed by the addition of a radioprotectant to said [¹⁸F]-fluciclatide; or
(iii) addition of a radioprotectant to [¹⁸F]-fluciclatide; or
(iv) combinations of (i), (ii) and (iii),
wherein said precursor is of Formula II:

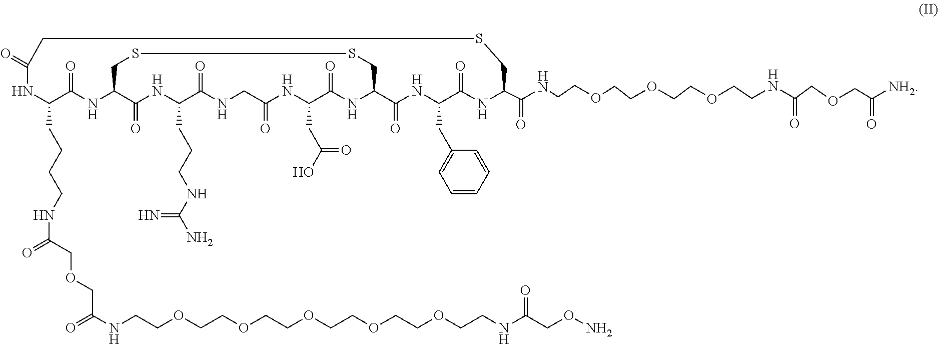 [SMILES: N=C(N)NCCC[C@@H]1NC(=O)[C@@H]2CSSC[C@H](NC(=O)[C@H](CC(=O)O)NC(=O)CNC1=O)C(=O)N[C@@H](Cc1ccccc1)C(=O)N[C@H](C(=O)NCCOCCOCCOCCNC(=O)COCC(N)=O)CSCC(=O)N[C@@H](CCCCNC(=O)COCC(=O)NCCOCCOCCOCCOCCOCCNC(=O)CON)C(=O)N2]

6. The method of claim 5, where the radioprotectant is sodium para-aminobenzoate.

7. The method of claim 5 which is carried out using an automated synthesizer apparatus.

8. The method of claim 7, where the automated synthesizer apparatus comprises a single use cassette, and said cassette comprises either:
(i) separate supplies of a precursor and a radioprotectant; or
(ii) a precursor and a radioprotectant, provided together as a composition.

9. A single use, cassette which comprises either:
(i) separate supplies of a precursor and a radioprotectant; or
(ii) a precursor and a radioprotectant, provided together as a composition;
wherein said radioprotectant is chosen from para-aminobenzoic acid or a salt thereof with a biocompatible cation, and wherein said precursor is of Formula II:

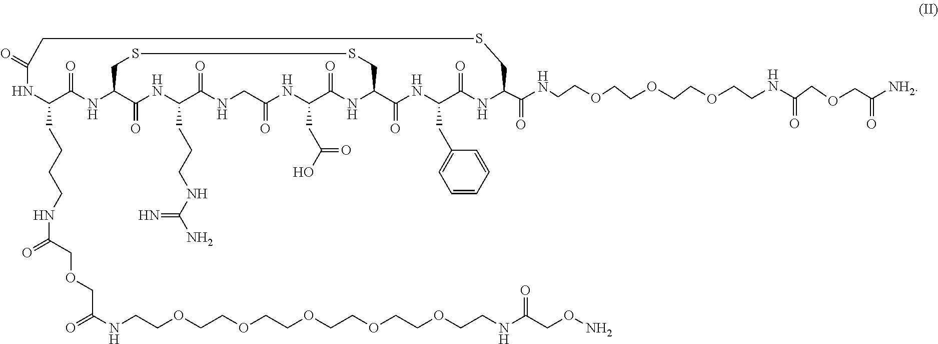 [SMILES: N=C(N)NCCC[C@@H]1NC(=O)[C@@H]2CSSC[C@H](NC(=O)[C@H](CC(=O)O)NC(=O)CNC1=O)C(=O)N[C@@H](Cc1ccccc1)C(=O)N[C@H](C(=O)NCCOCCOCCOCCNC(=O)COCC(N)=O)CSCC(=O)N[C@@H](CCCCNC(=O)COCC(=O)NCCOCCOCCOCCOCCOCCNC(=O)CON)C(=O)N2]

10. The cassette of claim 9, where the radioprotectant is provided as a solution.

11. The cassette of claim 9, where the radioprotectant is sodium para-aminobenzoate.

12. A method of stabilising a [¹⁸F]-fluciclatide radiopharmaceutical composition which comprises the use of para-aminobenzoic acid or a salt thereof with a biocompatible cation.

13. The method of claim 12, where the radioprotectant is sodium para-aminobenzoate.

14. A method of imaging of the mammalian body which comprises imaging a mammal which had previously been administered with the radiopharmaceutical composition of claim 1.

15. The method of claim 14, where the mammal is suffering from a disease in which integrins are abnormally expressed.

16. A method of diagnosis of the mammalian body which comprises the method of imaging of claim 14.
